(19) United States
(12) Patent Application Publication
(10) Pub. No.: US 2005/0100561 A1
(43) Pub. Date: May 12, 2005

(54) Use of topical compositions for the control of microbial diseases of the nail

(76) Inventors: Ronald James Sheen, Cottesloe (AU); Tom Anthony Langley, Nedlands (AU)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/414,745

(22) Filed: Apr. 16, 2003

Related U.S. Application Data
(60) Provisional application No. 60/373,239, filed on Apr. 17, 2002.

(51) Int. Cl. A61K 9/00

(52) U.S. Cl. 424/400

(57) ABSTRACT

A method of treating or preventing a microbial disease of a subject's nail, such as onychomycosis, the method comprising the step of topically administering to the nail an effective amount of copper silicate.

### FIELD OF THE INVENTION

The present invention relates to the use of copper silicate for treating and preventing microbial infections and diseases and in particular fungal diseases of the nail such as onychomycosis. The present invention also relates to copper silicate compositions adapted for topical administration.

### BACKGROUND ART

Microbial diseases of the nail are a considerable public health concern. Whilst there are diseases and disorders caused by bacteria, the most prevalent and clinically significant nail diseases are caused by fungi.

Onychomycosis is a fungal disease of nails caused by a range of fungi, principally dermatophytes—Trichophyton species such as Trichophyton mentagrophytes and Trichophyton rubrum, but also Trichophyton megninii, Trichophyton schoenleinii and Trichophyton tonsurans and Candida species, such as C. albicans. Infection is of the nail unit (the nail matrix, bed or plate) and although not life threatening it can cause inconvenience, pain, discomfort and sometimes serious physical and occupational limitations.

There is presently a range of oral medications available for treating onychomycosis. However, these are generally expensive, suffer from limited effectiveness, require close monitoring from practitioners and can cause serious side effects such as liver and heart damage.

Whilst there is a clear need for an effective topical agent for treating onychomycosis, the agents presently available have limited efficacy. The most widely used topical agent is Penlac®. However, the clinical studies concerning this agent report low efficacy.

The main difficulty faced when developing an effective topical agent for the nail is that the nail plate is thick, hard and dense, and represents a formidable barrier for drugs preventing them from reaching the infection site in a therapeutically required quantity. Adding to this problem is the fact that onychomycosis is associated with a thickening of the nail plate. To overcome this problem prior art topical agents for treating onychomycosis have incorporated agents that increase the permeability of the nail plate. However, these topical agents contain multiple agents i.e. a softener and an anti-fungal and thus are relatively expensive to produce. Furthermore, such agents have met with limited success.

The present invention seeks to overcome the above problems by providing a safe and effective topical anti-microbial that is capable of treating microbial infections of the nail.

### SUMMARY OF THE INVENTION

The present invention provides a method of treating or preventing microbial infection of a subjects nail, the method comprising the step of topically administering to said nail an effective amount of copper silicate.

The infections treated or prevented by the method of the present invention may manifest themselves into disease states that may also be treated according to the present invention. Thus, the present invention also provides a method of treating or preventing a microbial disease of a subject's nail, the method comprising the step of topically administering to the nail an effective amount of copper silicate.

The present invention also provides a composition adapted for topical administration comprising an anti-microbial effective amount of copper silicate.

### BRIEF DESCRIPTION OF THE FIGURES

FIG. 1 is a series of photos showing the time course of treatment (Day 0: untreated, week 6 and week 12) of a dermatophyte infection of a first subject's nail, according to one exemplary method of the present invention;

FIG. 2 is a series of photos showing the time course of treatment (Day 0: untreated, week 6 and week 12) of a dermatophyte infection of a second subject's nail, according to one exemplary method of the present invention;

FIG. 3 is a series of photos showing the time course of treatment (Day 0: untreated and week 6) of a dermatophyte infection of a third subject's nail, according to one exemplary method of the present invention;

FIG. 4 is a series of photos showing the time course of treatment (Day 0: untreated and week 6) of a dermatophyte infection of a fourth subject's nail, according to one exemplary method of the present invention;

FIG. 5 is a series of photos showing the time course of treatment (Day 0: untreated, week 12 and week 12—post-treatment) of a dermatophyte infection of a fifth subject's nail, according to one exemplary method of the present invention;

FIG. 6 is a series of photos showing the time course of treatment (Day 0: untreated, week 6, week 26 and week 12—post-treatment) of a dermatophyte infection of a sixth subject's nail, according to one exemplary method of the present invention; and

FIG. 7 is a series of photos showing the extended time course of treatment (Day 0: untreated, week 6, week 26 and week 12—post-treatment) of a dermatophyte infection of the second subject's nail, according to one exemplary method of the present invention.

### DETAILED DESCRIPTION OF THE INVENTION

The present invention provides a method of treating or preventing microbial infection of a subject's nail, the method comprising the step of topically administering to said nail an effective amount of copper silicate.

For the purposes of the present invention the term “topically administering” includes, but is not limited to, application through the nail plate with a device adapted to perforate the nail plate and application between the nail plate and nail bed with a device adapted to be inserted therebetween.

For the purposes of the present invention the term “nail” refers to one or more of the elements of the nail unit (the nail matrix, bed or plate) and also includes toe and fingernails and animal nail like features such as hooves, trotters and claws.

Surprisingly, it has been found that the copper silicate administered to a nail according to the present invention delivers either an effective anti-microbial dose to the infected area or a dose sufficient to prevent infection with a microbial nail pathogen. In this regard, the applicant has discovered that upon administration of the copper silicate to the nail, therapeutically effective amounts of the copper are able to reach the site of infection. The precise mode of this effect has not been established conclusively, but the Applicant is aware that the copper silicate persists at the site of application and/or the site of infection.

Topical administration may be achieved in any one of a number of ways. Preferably, the compound is applied to the nail by painting, wiping, dabbing or spraying the nail with the compound. Alternatively, topical administration may be achieved by submerging the nail in a copper silicate solution or contacting the nail with a means for dispensing an effective amount of copper silicate. Dispensing means can be varied and include slow release carriers and materials impregnated with copper silicate such as patches, bandages, cotton wool and gauze. Preferably, the dispensing means is adapted to be removably fixed to the nail to deliver the copper silicate over a predetermined time and/or at a predetermined dose.

The methods of the present invention may be used to treat microbial, such as fungal, infection in any animal and more particularly any mammal. However, preferably, the subject is human. Thus the present invention also provides for the use of copper silicate for preparing a medicament for treating microbial infections such as fungal infections including onychomycosis.

It is common for nails to be infected with a range of microbes such as bacteria and fungi (including yeast). Thus, the microbial infection may be bacterial. However, the method of the present invention is particularly useful for treating fungal infections.

The amount of copper silicate applied in the method of the present invention will be sufficient to effectively treat or prevent the infection and thus will necessarily vary depending at least on the severity and type of the infection, the strength of the composition and the manner in which individual patients respond to the treatment. However, preferably a composition containing 1-100 μg of copper is applied to each infected nail, more preferably a composition containing 2-50 μg of copper is applied to each infected nail and even more preferably, a composition containing 5-20 μg of copper is applied to each infected nail. In one particular form of the invention a composition containing at least about 10 μg of copper is applied to each infected nail.

Similarly, the frequency with which, and the duration for which, the copper silicate is applied will be sufficient to effectively treat or prevent the infection and thus will also vary depending at least on the severity and type of infection, the strength of the composition and the manner in which individual patients respond to the treatment. Preferably, the copper silicate is applied at least once, twice or three times a day for at least 1, 3, 6, 12, 26, 30, 40, 45, 48 or 52 weeks. Alternatively, it may be applied once every 2-14 days for as long as necessary to treat or prevent infection.

Thus, the present invention also provides a method of treating or preventing microbial infection of a subject's nail, the method comprising the step of repeatedly topically administering to said nail an effective amount of copper silicate.

As indicated above the method of the present invention may be applied to treat fungal infections. In this regard, fungal infections which may be treated according to the present invention include infections from dermatophytes, such as Trichophyton species including Trichophyton mentagrophytes (including varieties mentagrophytes and interdigitales), Trichophyton rubrum, Trichophyton megninii, Trichophyton schoenleinii, Trichophyton tonsurans; yeasts, such as Candida species, most notably Candida albicans, Candida glabrata, Candida ciferrii; Microsporum species including Microsporum canis and Microsporum gypseum; Malassezia species including Malassezia furfur and Malassezia sympodialis; Epidermophyton species such as Epidernophyton floccosum; Scopularosis species; Acremonium species and Aspergillus species.

Thus, the present invention also provides a method of treating or preventing infection of a subject's nail with one or more of: dermatophytes, such as Trichophyton species including Trichophyton mentagrophytes (including varieties mentagrophytes and interdigitales), Trichophyton rubrum, Trichophyton megninii, Trichophyton schoenleinii, Trichophyton tonsurans; yeasts, such as Candida species, most notably Candida albicans, Candida glabrata, Candida ciferrii; Microsporum species including Microsporum canis and Microspomm gypseum; Malassezia species including Malassezia furfur and Malassezia sympodialis; Epidermophyton species such as Epidermophyton floccosum; Scopularosis species; Acremonium species and Aspergillus species, the method comprising the step of topically administering to said nail an effective amount of copper silicate.

Fungal nail infections that may be treated using the methods of the present invention are usually characterized by tarnished white, yellowed, or blackened nails. The nails may pull away from the pink nail bed along the sides or outer edges, and infections are usually exacerbated by hot, damp conditions, such as inside footwear or in environments where hands or feet are continually exposed to moisture. The fungal infections can spread from toe to toe, foot to foot, and foot to hand.

Methods of Treating or Preventing Microbial Diseases

The present invention also provides a method of treating or preventing a microbial disease of a subject's nail, the method comprising the step of topically administering to the nail an effective amount of copper silicate.

Whilst the method of the present invention may be applied to treat a wide range of microbial diseases of the nail it is particularly useful for treating fungal diseases of the nail. Thus, the present invention also provides a method of treating or preventing a fungal disease of a subject's nail, the method comprising the step of topically administering to the nail an effective amount of copper silicate.

Specific fungal diseases that can be treated by the methods of the present invention include onychomycosis such as distal subungual onychomycosis, superficial white onychomycosis, proximal white subungual onychomycosis; total secondary dystrophic onychomycosis; and total dystrophic primary onychomycosis.

In some situations, it may be desirable to combine the method of treating or preventing a fungal disease according to the present invention with other therapies. Thus, the present invention also provides a method of treating or preventing a fungal disease of a subject's nail, the method comprising the step(s) of topically administering to said nail an effective amount of copper silicate and (ii) administering to said subject an effective amount of at least one other anti-fungal agent.

The other anti-fungal agent may be varied and includes, for example, one or more orally administrable anti-fungal agents selected from the group consisting of: miconazole, ketoconazole, itraconazole, fluconazole, econazole, ciclopirox, oxiconazole, clotrimazole, terbinafine, naftifine, pharmaceutically acceptable salts thereof and stereoisomers thereof, Lamisil™, Sporanox™ and Diflucan™.

The present invention also provides for the use of copper silicate for preparing a medicament for treating or preventing a disease caused by a fungal infection.

Topical Formulations

The present invention also provides a composition adapted for topical administration to a nail comprising an anti-microbial effective amount of copper silicate.

The form of the composition of the present invention may also be varied provided it retains its anti-microbial properties. Preferably, the composition is a solution. However, the composition may also be in solid form provided it is properly formulated. In this regard, the composition could comprise copper silicate in the form of a micronized solid such as chrysocolla.

When the copper silicate is provided in the form of a solution, it preferably is provided as an aqueous acidified solution. Acidified solutions are particularly preferred because copper silicate is more soluble at acidic pH. Particularly preferred pHs are 3-6, 4-6 and 5-6. In one example, the copper silicate is prepared according to the methods described and claimed in U.S. Pat. No. 5,474,972.

The composition adapted for topical administration may be in the form of any one of the following: solution, lotion, suspension, emulsion, cream, gel, ointment, liniment and salve. Particularly preferred forms are ointments, creams or gels.

Ointments generally are prepared using either (1) an oleaginous base, i.e., one consisting of fixed oils or hydrocarbons, such as white petroleum or mineral oil, or (2) an absorbent base, i.e., one consisting of an anhydrous substance or substances that can absorb water, for example anhydrous lanolin. Customarily, following formation of the base, whether oleaginous or absorbent, the active ingredient is added to an amount affording the desired concentration.

Creams are oil/water emulsions. They consist of an oil phase (internal phase), comprising typically fixed oils, hydrocarbons and the like, waxes, petroleum, mineral oil and the like and an aqueous phase (continuous phase), comprising water and any water-soluble substances, such as added salts. The two phases are stabilised by use of an emulsifying agent, for example, a surface active agent, such as sodium lauryl sulfate; hydrophilic colloids, such as acacia colloidal clays, veegum and the like. For the purposes of the present invention, the compound may be added to the water phase prior to formation of the emulsion, in an amount to achieve the desired concentration.

Gels comprise a base selected from an oleaginous base, water, or an emulsion-suspension base. To the base is added a gelling agent that forms a matrix in the base, increasing its viscosity. Examples of gelling agents are hydroxypropyl cellulose, acrylic acid polymers and the like. For the purposes of the present invention the compound may be added to the formulation at the desired concentration at a point preceding addition of the gelling agent.

The compositions of the present invention may be produced by dissolving or combining the copper silicate in an aqueous or non-aqueous carrier. In general, any liquid, cream, or gel, or similar substance that does not appreciably react with the silicate or any other active ingredient that may be introduced and which is non-irritating is suitable.

Thus, the present invention also provides a method of producing copper silicate adapted for topical administration comprising the step of dissolving or combining the copper silicate in an aqueous or non-aqueous topical carrier.

The composition of the present invention may further comprise an auxiliary agent such as any one or more of: preservatives, stabilizers, emulsifiers, wetting agents, fragrances, colouring agents, odour controllers and thickeners such as natural gums.

The concentration of the copper in the composition may be varied depending at least on the severity and type of infection that the composition is to be used to treat or prevent. However, preferably, the concentration of the copper is approximately 100 mg/L-10 g/L (as Cu). More preferably, the copper concentration is to a final concentration of approximately 1 g/L-5 g/L or 2 g/L-3 g/L (as Cu).

The present invention will now be described with reference to the following examples. The description of the examples is in no way to limit the generality of the preceding description.

### EXAMPLES

### Example 1

### In Vitro Funigicidal Activity of a Copper Silicate Solution

Trials were carried out to assess the fungicidal activity of copper silicate against a range of fungi. The results of the study are set out in Tables 1a and 1b hereunder.

TABLE 1a
Minimum inhibitory (MIC) and fungicidal (MFC) concentrations of a
copper silicate solution (ppm Cu) for Candida spp., Malassezia
spp. and various dermatophytes.

| Organism | N | MIC* | MFC* |
|---|---|---|---|
| Candida albicans | 8 | 128 | 1024 |
| Candida ciferrii | 2 | 128 | 128 |
| Candida glabrata | 2 | 256 | 256 |
| Malassezia furfur | 6 | 512 | >1024 |
| Malassezia sympodialis | 7 | 512 | >1024 |
| Epidermophyton | 10 | 256 | 256 |
| floccosum | | | |
| Microsporum canis | 10 | 128 | 256 |
| Microsporum gypseum | 10 | 128 | 256 |
| Trichophyton | 10 | 256 | 256 |
| mentagrophytes var | | | |
| interdigitales | | | |
| Trichophyton | 10 | 256 | 256 |
| mentagrophytes var | | | |
| mentagrophytes | | | |
| Trichophyton rubrum | 10 | 128 | 256 |
| Trichophyton tonsurans | 10 | 128 | 256 |

*Modal values

TABLE 1b
Minimum Inhibitory Concentration (MIC) and Minimum Fungicidal
Concentration (MFC) of copper silicate (% w/w as Cu) for various
dermatophytes and non-dermatophytes that cause onychomycosis,
and Candida spp.

| Species | n | MIC* | MFC* |
|---|---|---|---|
| Trichophyton rubrum | 26 | 0.0175 | 0.0350 |
| T. mentagrophytes | 19 | 0.0175 | 0.0350 |
| var interdigitale | | | |
| T. mentagrophytes | 12 | 0.0175 | 0.0350 |
| T. tonsurans | 10 | 0.0350 | 0.0350 |
| Epidermophyton floccosum | 11 | 0.0350 | 0.0350 |
| Microsporum canis | 9 | 0.0350 | 0.0700 |
| M. gypseum | 9 | 0.0350 | 0.1400 |
| Aspergillus flavus | 12 | 0.1400 | >0.1400 |
| A. fumigatus | 12 | 0.0350 | >0.1400 |
| A. niger | 14 | 0.0350 | 0.1400 |
| A. terrus | 7 | 0.0700 | >0.1400 |
| Fusarium spp. | 16 | 0.0700 | 0.0700 |
| Scopulariopsis spp. | 5 | 0.0350 | >0.1400 |
| Scedosporium spp. | 5 | 0.0350 | >0.1400 |
| Candida albicans | 10 | 0.0350 | 0.0700 |
| C. tropicalis | 10 | 0.0350 | 0.0700 |
| C. parasilosis | 10 | 0.0350 | 0.0350 |
| C. glabrate | 4 | 0.0350 | 0.1400 |
| C. pseudotropicalis (kefyr) | 1 | 0.0350 | 0.0350 |
| C. krusei | 1 | 0.0175 | 0.0175 |

*Modal Values

### Example 2

### In Vitro Bactericidal Activity of a Copper Silicate Solution

TABLE 2
Minimum inhibitory concentrations (MIC) of copper silicate solution
(ppm Cu) for a variety of bacteria

| Organisms | n | Range | MIC⁹⁰ |
|---|---|---|---|
| Met^{R} - Staphylococcus aureus | 13 | 256-512 | 512 |
| Met^{S} - Staphylococcus aureus | 9 | 256-512 | NA |
| Propionibacterium acne | 19 | 4-256 | 256 |
| Staphylococcus saprophyticus | 4 | 1024 | NA |
| Streptococcus pneumoniae | 27 | 128-512 | 512 |
| Streptococcus pyogenes | 11 | 256-1024 | 512 |
| Streptococcus spp. | 8 | 128-1024 | NA |
| Acinetobacter spp. | 13 | 512-1024 | 1024 |
| Enterobacteriaceae* | 113 | 1024->1024 | >1024 |
| Enterococcus faecalis | 12 | 64-256 | 256 |
| Enterococcus faecium | 2 | 128-256 | NA |
| Morganella morganii | 7 | >1024 | NA |
| Moraxella catarrhalis | 3 | <6 | NA |
| Pseudomonas aeruginosa | 13 | 1024->1024 | >1024 |
| Stenotrophomonas maltophilia | 12 | 1024->1024 | >1024 |

Enterobacteriaceae spp. include Citrobacter freundii (n = 11), Enterobacter aerogenes (n = 13), Enterobacter cloacae (n = 13), Escherichia coli (n = 25), Klebsiella oxytoca (n = 10), Klebsiella pneumoniae (n = 24), Proteus mirabilis (n = 12) and Providencia spp.(n = 5)

### Example 3

### Treatment of Onychomycosis with Copper Silicate

This study evaluates copper silicate as a topical fungicide as a treatment of onychomycosis and compares a solution and cream formulation.

Materials/Methods

(A) Fungicidal Solution (CSSOL1)

Ingredients

Sodium silicate solution (30% SiO₂, 10% Na₂O)
Empimin LSM 30AU™—Huntsman International LLC (sodium alkyl ether sulfate)
Copper sulfate pentahydrate
Acetic Acid (90% solution)
Water (deionised)
   Technical Instructions

1. The solution is prepared as two separate parts (A and B) as follows:

Part A Silicate Solution

| Product | Weight (g) |
|---|---|
| Water | 56.7 |
| Sodium silicate solution | 60.8 |
| (30% SiO₂, 10% Na₂O) | |
| Empimin LSM 30AU ™ | 2.5 |
| Total | 120 g (˜100 ml) |

Method: Add the sodium silicate solution (30% SiO₂, 10% Na₂O) and empimin LSM 30AU™ to the water with stirring. Filter.

Part B. Copper Solution

| Product | Weight (g) |
|---|---|
| Water | 88.2 |
| Copper sulfate pentahydrate | 11.5 |
| Acetic acid (90%) | 9.4 |
| Total | 109.1 g (˜100 ml) |

Method: Add the copper sulfate pentahydrate to the water with stirring and continue stirring until a clear solution is obtained. Add the acetic acid with stirring. Filter.

2. The final solution is then produced by dilution of the combined parts.

| Product | Weight (g) |
|---|---|
| Water | 880.0 |
| Part A | 24.0 |
| Part B | 109.1 |
| Total | 1013.1 (˜1000 mL) |

Method: Add Part A to the water with stirring. Continue to stir while adding Part B. Filter.

The final product should be a light blue, clear solution with an acrid odour.

Product Specifications:

pH: 3.3-3.7
Copper content: 2.8+/−0.28 g/L as Cu
   (B) Copper Silicate Solution Concentrate for Use in Creams (CSC1)
   Ingredients Required
Sodium silicate solution (30% SiO₂, 10% Na₂O)
Copper sulfate pentahydrate
Acetic Acid (90% solution)
Water (deionised)
   Technical Instructions:

1. The solution is prepared as two separate parts (A and B) as follows:

Part A. Silicate Solution

| Product | Weight (g) |
|---|---|
| Water | 59.2 |
| Sodium silicate solution | 60.8 |
| (30% SiO₂, 10% Na₂O) | |
| Total | 120 g (˜100 mL) |

Method: Add the sodium silicate solution (30% SiO₂, 10% Na₂O) to the water with stirring. Filter.

Part B. Copper Solution

| Product | Weight (g) |
|---|---|
| Water | 88.2 |
| Copper sulfate pentahydrate | 11.5 |
| Acetic acid (90%) | 9.4 |
| Total | 109.1 g (˜100 mL) |

Method: Add the copper sulfate pentahydrate to the water with stirring and continue stirring until a clear solution is obtained. Add the acetic acid with stirring. Filter.

2. The final concentrated solution is then produced by dilution of the combined parts.

CSC1 Concentrate Solution

| Product | Weight (g) |
|---|---|
| Water | 130.0 |
| Part A | 24.0 |
| Part B | 109.1 |
| Total | 263.1 (˜250 mL) |

Method: Add Part A to the water with stirring. Continue to stir while adding Part B. Filter.

The final product should be a light blue, clear solution with an acrid odour.
Copper content: 11.2 g/L as Cu
   (C) Fungicidal Cream

1. Formulation

| Ingredient | | % wt |
|---|---|---|
| Phase A. | Water | 42.77 |
| | Copper silicate conc. | 20.00 |
| | (CSC1 - pH adjusted to 5.0 | |
| | with triethanolamine) | |
| | Propylene glycol | 5.00 |
| | Germaben IIE ™ (Sutton Laboratories | 0.50 |
| | Inc) | |
| | Brij 78 ™ (Atlas Powder Company) | 3.00 |
| | Sorbic Acid | 0.10 |
| | Red no. 33 Solution (0.02%) | 0.20 |
| Phase B. | Propyl paraben | 0.20 |
| | Tween 60 ™ (Atlas Powder Company) | 2.56 |
| | Stearyl alcohol | 3.61 |
| | Ceteth-20 | 3.00 |
| | Cetyl alcohol | 2.56 |
| | Cetearyl alcohol | 2.00 |
| | Myristyl myristate | 2.00 |
| | PEG-4 Olivate | 5.00 |
| | Octyl palmitate | 4.00 |
| | Glyceryl stearate (and) | 3.00 |
| | PEG-100 stearate | |
| | 20% Titanium dioxide paste | 0.50 |
| Total | | 100.00 |

2. Method

a. Into the main mixing vessel which should be fitted with a variable speed “Silverson” mixer and a sweep-blade contra-rotating agitator, weigh the ingredients of Phase B. Stir until uniform. Adjust temperature to 75° C.

b. Into a separate vessel weigh the phase A ingredients, mix with a propeller stirrer until all solids are dissolved. Adjust temperature to 75° C.

c. Add phase A to phase B, using the “Silverson” mixer to form a smooth even emulsion.

d. Switch on the sweep-blade agitator and begin cooling the batch.

e. Continue cooling and mixing until the temperature is 35° C.

f. pH should be 5.3-5.5.

Subjects, not already receiving anti-fungal treatment, were assessed for clinical diagnosis of onychomycosis and photographs and nail clippings were taken. Those testing positive for onychomycosis were admitted to the trial.

The anti-fungal composition (cream or solution) was applied to the infected nail twice daily for the duration of the treatment.

Estimates of the nail area involved were made during clinical visits and photographs were taken. Nail clippings were collected for KOH and dermatophyte culture. Subjects were assessed by a clinician for onychomycosis using the following criteria:
“Not effective” is ≧10% nail involvement with KOH positive and culture positive;
“Mycological cure” KOH negative and culture negative;
“Effectively treated” is ≦10% nail involvement, with KOH and culture negative; and
“Complete cure” is clinical clearance of the target nail together with negative KOH and culture.
   Results

The results presented hereunder are interim results of a 12 month study. Patients entered the trial and commenced their treatment at different dates. Of the 18 patients enrolled in the trial, 13 had reached 6 weeks of treatment, and of these, 10 had reached 12 weeks of treatment, at the time of reporting these results. Patients were assessed at 0 (commencement). 6 weeks and 12 weeks. The results of these assessments are tabulated below in Table 3. No differences were noted in efficacy between the cream and solution at any of the assessments.

TABLE 3

| | % of Patients | | |
|---|---|---|---|
| | Day 0 | 6 Weeks | 12 Weeks |
| Criteria | (n = 18) | (n = 13) | (n = 10) |
| Mycology (KOH | 0 | 53 | 80 |
| negative or culture | | | |
| negative) | | | |
| Mycology (KOH | 0 | 0 | 20 |
| negative and culture | | | |
| negative) | | | |
| Clinical - New growth | 0 | 61 | 70 |
| clear | | | |
| Clinical - 100% clear | 0 | 0 | 30 |
| Complete cure | 0 | 0 | 20 |
| (KOH negative, culture | | | |
| negative and 100% | | | |
| clear) | | | |

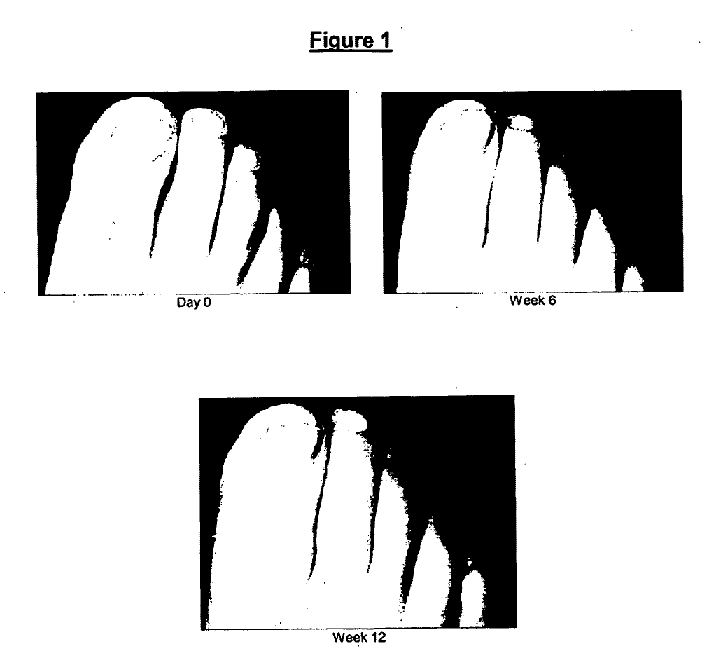

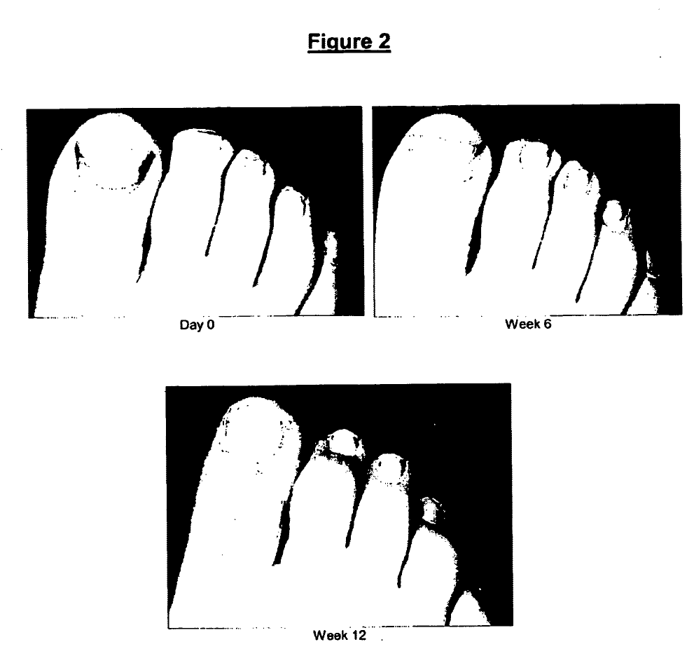

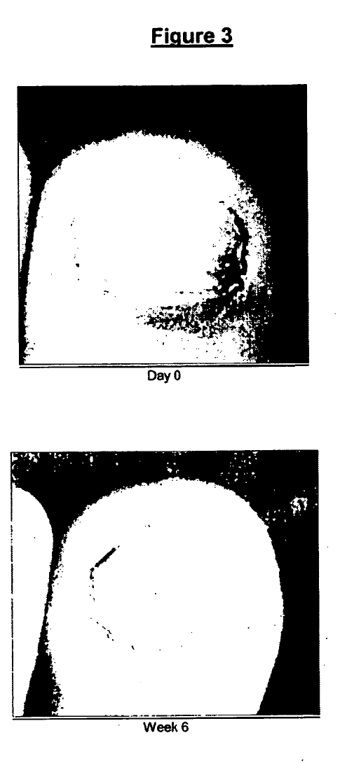

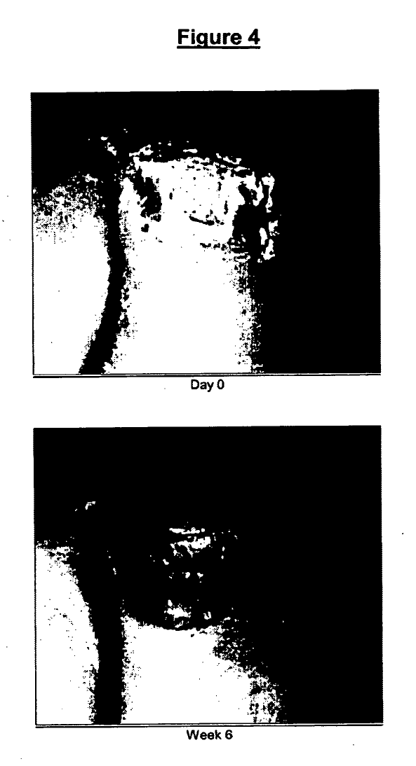

The effectiveness of the treatment method in the example is exemplified in FIGS. 1-4 that contain photographs of patient's nails treated in the trial.

No adverse reactions were reported.

### Example 4

### Treatment of Onychomycosis with Copper Silicate

The trial reported in Example 3 was continued and the updated results are set out hereunder in Tables 4, 5 and 6 which list the clinical, mycological and efficacy measures, respectively.

TABLE 4

| | Treatment time | | | | Post-treatment | |
|---|---|---|---|---|---|---|
| | (N = 18) | | | | (N = 18) | |
| | | | Week | Week | Week | Week |
| | Day 0 | Week 6 | 12 | 26 | 13 | 26 |
| % Nail infected | | | | | | |
| 0% (clear) | 0 | 0 | 3 | 3 | 3 | 3 |
| 1-10% | 1 | 2 | 1 | 1 | 1 | 2 |
| 11-50% | 5 | 5 | 4 | 8 | 6 | 6 |
| >50% | 12 | 11 | 10 | 6 | 8 | 7 |
| New growth | | | | | | |
| Clear | 10 | 11 | 16 | 16 | 15 | 15 |
| Not clear | 8 | 7 | 2 | 2 | 3 | 3 |

Note:
One of the “not clear” patients had a damaged nail plate and was always assessed as “not clear”.

TABLE 5

| | Treatment time | | | | Post-treatment | |
|---|---|---|---|---|---|---|
| | (N = 18) | | | | (N = 18) | |
| | | | Week | Week | Week | Week |
| | Day 0 | Week 6 | 12 | 26 | 13 | 26 |
| KOH | | | | | | |
| Positive | 17 | 14 | 14 | 12 | 14 | 12 |
| Negative | 1 | 4 | 4 | 6 | 4 | 6 |
| Dermatophyte culture | | | | | | |
| Positive | 18 | 8 | 7 | 6 | 8 | 9 |
| Negative | 0 | 10 | 11 | 12 | 10 | 9 |
| Infecting organism | | | | | | |
| T. rubrum | 12 | 7 | 6 | 5 | 7 | 8 |
| T. mentagrophytes v. | 6 | 1 | 1 | 1 | 1 | 1 |
| interdigitale | | | | | | |

Note:
Between 39 weeks and 52 weeks (26 weeks post treatment) another patient who had been assessed as culture negative reverted to culture positive with growth of T. rubrum being recorded. All 3 “clinical cure” patients were infected with T. mentagrophytes v. interdigitale.

TABLE 6

| | Treatment time | | | Post-treatment | |
|---|---|---|---|---|---|
| | Week 6 | Week 12 | Week 26 | Week 13 | Week 26 |
| Outcomes | (N = 18) | (N = 18) | (N = 18) | (N = 18) | (N = 18) |
| Complete | 0 | 2 | 3 | 3 | 3 |
| cure | | | | | |
| Effective | 2 | 3 | 3 | 3 | 4 |
| treatment | | | | | |
| Mycological | 4 | 3 | 5 | 3 | 4 |
| cure | | | | | |
| Mycological | 6 | 8 | 7 | 7 | 5 |
| change | | | | | |
| Not | 8^{#} | 7^{@} | 6^{@} | 8^{@} | 9^{@} |
| effective | | | | | |

^{#}includes 2 patients that are KOH negative
^{@}includes 1 patient that is KOH negative

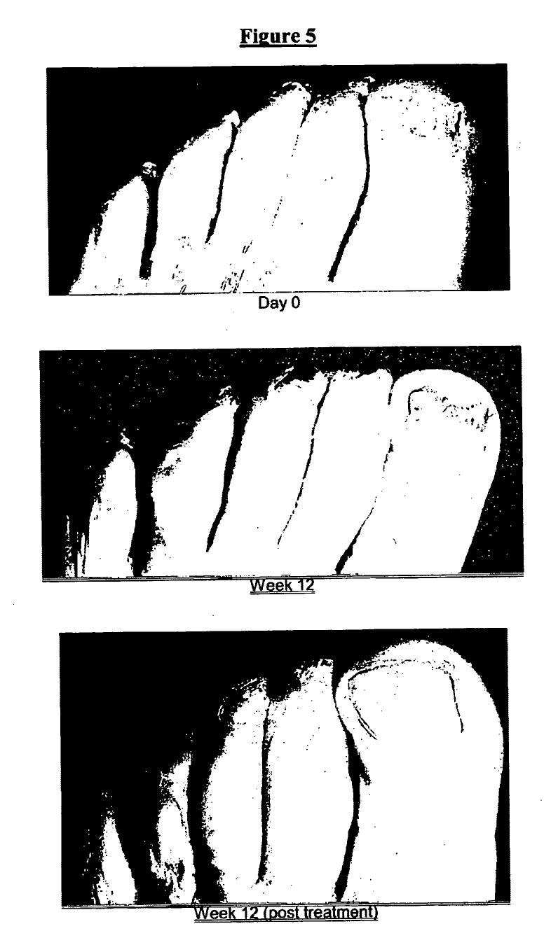

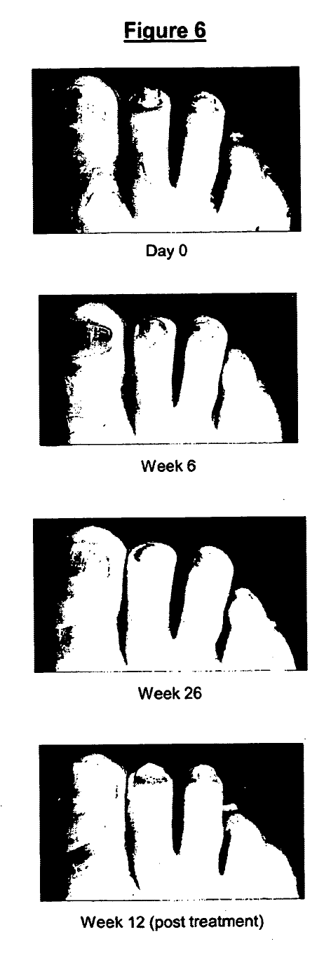

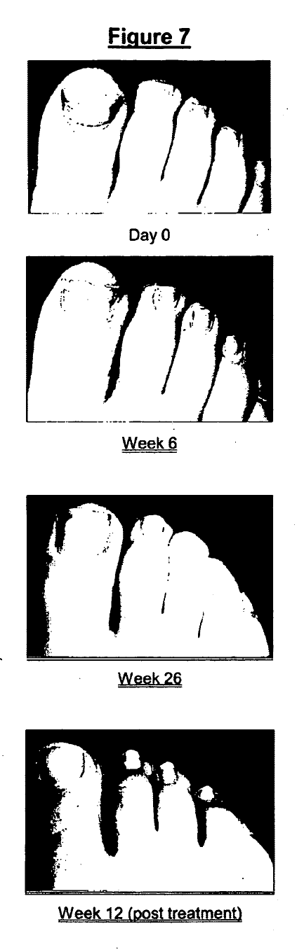

The effectiveness of the treatment method in the example is exemplified in FIGS. 5-7 that contain photographs of patient's nails treated in the trial.

No adverse reactions have been reported.

### SUMMARY

At week 26, 3 patients achieved the primary endpoint of “complete cure” and maintained their status 26 weeks post-treatment at 52 weeks. By 26 weeks post treatment 4 (22%) patients were “effectively treated”, an increase by one patient over 26 and 39 weeks which only included the 3 “complete cure” patients.

“Mycological cure” was achieved in 5 (28%) patients after 26 weeks, but 2 of these had reverted to KOH positive at 39 weeks. At 52 weeks the “mycological cure” rate was 22%.

“Mycological change”, or the change from culture positive to culture negative, was achieved by another 7 patients at weeks 26 and 39. This gave a total of 12 culture negative patients at 26 weeks and 10 at 39 weeks.

In total, 67% of patients were culture negative at the end of treatment, 55% at 13 weeks post-treatment, and 50% (9 patients) at the end of the trial.

The primary and secondary endpoints of “complete cure” and “effective treatment” are both dependent upon the mycological cure rate. “Mycological cure” is defined as both KOH and culture negative, but patients may not become KOH negative until the diseased nail has grown out. It is usual for a great toenail to take 12-18 months to totally regrow. At the commencement of this trial, 12 of the 18 patients had >50% of their target nail infected, and of these 7 had cuticle (lunula) involvement. As a result of extensive disease, “mycological cure” and thus “effective treatment” and “complete cure” may not have been achievable by these patients in the time allotted to this study.

The treatment time of 26 weeks is considerably shorter than for most topicals that are applied for a minimum of 48 weeks. It is evident from this trial that patients with >50% of the nail plate diseased would have benefited from an extended treatment time. The results at 26 weeks show that many (50%) had significant clinical improvement with a reduction in the extent of their disease to <50% of the nail plate.

The present invention includes any and all modifications and adaptations apparent to one skilled in the art.

Throughout the specification, unless the context requires otherwise, the word “comprise” or variations such as “comprises” or “comprising”, will be understood to imply the inclusion of a stated integer or group of integers but not the exclusion of any other integer or group of integers.

1. A method of treating or preventing microbial infection of a subject's nail, the method comprising the step of topically administering to said nail an effective amount of a copper silicate composition.

2. A method according to claim 1 wherein the microbial infection is a fungal infection.

3. A method according to claim 1 wherein the subject is a mammal.

4. A method according to claim 3 wherein the subject is a human.

5. The use of copper silicate for preparing a medicament for treating a microbial nail infection.

6. The use of copper silicate for preparing a medicament for treating a fungal nail infection.

7. A method according to claim 1 wherein the effective amount of copper silicate composition includes approximately 1 to 100 μg of copper.

8. A method according to claim 1 wherein the effective amount of copper silicate composition includes approximately 2 to 50 μg of copper.

9. A method according to claim 1 wherein the effective amount of copper silicate composition includes 10 μg of copper.

10. A method according to claim 1 wherein the copper silicate composition is applied at least 1 to 3 times a day.

11. A method according to claim 1 wherein the copper silicate composition is applied at least once every 2 to 14 days.

12. A method of treating or preventing infection of a subject's nail with one or more of: dermatophytes, such as Trichophyton species including Trichophyton mentagrophytes (including varieties mentagrophytes and interdigitales), Trichophyton rubrum, Trichophyton megninii, Trichophyton schoenleinii, Trichophyton tonsurans; yeasts, such as Candida species, Candida albicans, Candida glabrata, Candida ciferrii; Microsporum species including Microsporum canis and Microsporum gypseum; Malassezia species including Malassezia furfur and Malassezia sympodialis; Epidermophyton species such as Epidermophyton floccosum; Scopularosis species; Acremonium species and Aspergillus species, the method comprising the step of topically administering to said nail an effective amount of a copper silicate composition.

13. A method of treating or preventing a microbial disease of a subject's nail, the method comprising the step of topically administering to the nail an effective amount of a copper silicate composition.

14. A method according to claim 13 wherein the microbial disease is selected from the group comprising: onychomycosis, distal subungual onychomycosis, superficial white onychomycosis, proximal white subungual onychomycosis; total secondary dystrophic onychomycosis; and total dystrophic primary onychomycosis.

15. A method of treating or preventing a fungal disease of a subject's nail, the method comprising the step(s) of topically administering to said nail an effective amount of a copper silicate composition and (ii) administering to said subject an effective amount of at least one other anti-fungal agent.

16. The use of copper silicate for preparing a medicament for treating or preventing onychomycosis.

17. A composition adapted for topical administration to a nail comprising an anti-microbial effective amount of copper silicate.

18. A composition according to claim 17 in the form of a solution.

19. A composition according to claim 18 in the form of an aqueous acidified solution.

20. A composition according to claim 17 in the form of a solid.

21. A method of producing a copper silicate composition adapted for topical administration comprising the step of dissolving or combining the copper silicate in an aqueous or non-aqueous topical carrier.

22. A composition according to claim 17 comprising approximately 100 mg/L to 10 g/L (as Cu).

23. A composition according to claim 17 comprising approximately 1 g/L to 5 g/L.

24. A composition according to claim 17 comprising approximately 2 g/L to 3 g/L (as Cu).

25. A method according to claim 1, 12, 13 or 15 wherein the copper silicate composition comprises a preservative.
